(19) United States
(12) Patent Application Publication
(10) Pub. No.: US 2013/0110232 A1
(43) Pub. Date: May 2, 2013

(54) COMPOSITE PRODUCT

(71) Applicant: France Chirurgie Instrumentation SAS (FCI), Paris (FR)

(72) Inventors: Christophe HUPIN, Salles (FR); Laurent FOUCHET, Prignac et Marcamps (FR); Marc DOLATKHANI, Cestas (FR)

(73) Assignee: FRANCE CHIRURGIE INSTRUMENTATION SAS (FCI), Paris (FR)

(21) Appl. No.: 13/653,631

(22) Filed: Oct. 17, 2012

(30) Foreign Application Priority Data
Oct. 28, 2011 (FR) 11 03298
Feb. 16, 2012 (FR) 12 00448

(51) Int. Cl. A61F 2/14; B32B 3/24; A61F 2/44; B05D 5/00

(52) U.S. Cl. 623/4.1; 427/243; 428/137; 442/1; 623/17.16

(57) ABSTRACT

A composite product, especially an implant, includes at least one base element or matrix of a first polymer material coated with a coating of a second polymer material or of a second material based on reactive prepolymers. There is at least one base element or matrix coated with the coating which exhibits openings passing through it from one side to the other. The softening temperature of the second polymer material or the reaction/crosslinking temperature of the second material based on reactive prepolymers being lower than the softening temperature of the first polymer material.

### TECHNICAL FIELD

The present invention relates to a composite product, especially an implant, for example an intraocular ball, which has good porosity, especially porosity permitting cell growth within the implant. The present invention relates also to a process for the production of a porous composite product of this type. The present invention relates also to the use of a composite product of this type in the medical field.

### BACKGROUND ART

Porous composite products which are used in the medical field, especially in the form of an implant, are already known from the prior art. There are known, especially, porous composites made of ceramics material. These porous composites made of ceramics material are relatively heavy, lack flexibility and are therefore uncomfortable for the wearer of the implant.

Also known from the prior art are porous composite products which are produced from layers of porous polymers which are fixed together. However, although these composite products have greater flexibility than composite products made of ceramics, as well as high porosity, they are complicated to produce and the risk of rupture between the separate entities forming the composite product is high, which results in a loss of the capabilities of the composites.

### DISCLOSURE OF THE INVENTION

The present invention aims to overcome the disadvantages of the prior art by proposing a porous composite product which is simple to produce and which, further, withstands use under high repeated stresses without any significant risk of its rupturing, which is particularly appropriate for medical use as an implant.

Accordingly, there is obtained a particularly strong composite product, the second material added to the base substrate having a cementing function in order to maintain the shape of the substrate. The effect of heating and cooling the second material is used to obtain the substrate in a specific shape.

According to a preferred embodiment of the invention, the matrix is constituted in the form of a mesh.

According to another preferred embodiment of the invention, the matrix is constituted by a perforated woven sheet or by a perforated nonwoven sheet.

According to a preferred embodiment of the invention, the composite product comprises a plurality of matrices of a first polymer material which are coated with a second polymer material and stacked one on top of the other.

According to another preferred embodiment of the invention, the composite product is constituted by a matrix of the first polymer material which is coated with the second polymer material and folded up, preferably several times.

Preferably, the plurality of stacked matrices or the folds of the folded matrix are fixed together, especially by hot moulding or thermoforming.

In that manner, according to the invention, there are obtained composite products, especially implants, which have high porosity and which are easy to produce and which, at the same time, withstand repeated stresses without breaking without warning.

Preferably, the softening temperature of the second polymer material is higher than 45° C., preferably higher than 50° C.

Preferably, the softening temperature of the first polymer material is higher than 90° C.

The present invention relates also to a process for the production of a porous composite product, especially a porous implant, as defined in claim 11, improvements being defined in sub-claims 12 and 13.

According to a preferred embodiment of the invention, before the thermoforming step, the coated base element is folded up in a plurality of folds, and then the thermoforming step is carried out in a mould or hot press, the shape of which corresponds to the composite product that is to be obtained.

According to another preferred embodiment of the invention, a plurality of base matrices of a first polymer material that are coated with the second polymer material is stacked, and hot pressing is carried out in a mould or in a hot press, the shape of which corresponds to that of the product that is to be obtained.

According to the invention, the second polymer is understood both as being a polymer material and as prepolymer materials.

### BRIEF DESCRIPTION OF THE DRAWINGS

FIG. 1 shows schematically the folding protocol of a preformed element cut from a coated PP mesh during the production process of porous balls of Example 1.

### PREFERRED EMBODIMENT FOR CARRYING OUT THE INVENTION

Shaping of the composite product is carried out by hot moulding or thermoforming at a temperature higher than the softening temperature (T_{soft}° C.) of the second polymer material Pᵤ or, in the case of the use of reactive prepolymers Pᵣ, at a temperature higher than their reaction/crosslinking temperature (T_{cross}° C.). That temperature must, in addition, be lower than the softening temperature of the first polymer material of the matrix (M), so that it retains its physical, chemical and mechanical integrity. These conditions imply that T_{soft}° C. (M)>T_{soft}° C. (Pᵤ)

or T_{soft}° C. (Pᵤ)<T° C._{moulding} or _{thermoforming}<T_{soft}° C. (M)
or that (T_{soft}° C.) (M)>T_{cross}° C. (Pᵣ)
or T_{cross}° C. (Pᵣ)≦T° C._{moulding} or _{thermoforming}<T_{soft}° C. (M)

Cooling of the mould or of the press to a temperature below the softening point of Pᵤ allows the device to be fixed and maintained in its shape, the matrix being imprisoned by interpenetration of the polymer (Pᵤ) which has returned to the vitreous or crystalline state or of the three-dimensional lattice constituted by the crosslinked prepolymers (Pᵣ). In addition, inasmuch as the implantable composite products are to operate at temperatures of from 35 to 42° C., another required condition in this process is that the polymer (Pᵤ) has a softening temperature higher than body temperature and at least equal to or higher than 45° C. and, preferably, equal to or higher than 50° C.

In the case of the use of prepolymers (Pᵣ), the matrix (M) is imprisoned in the three-dimensional lattice formed by (Pᵣ).

The matrices are selected as a function of a group of parameters such as chemical nature, biocompatibility, biodegradability or non-biodegradability in the body, mechanical and thermomechanical characteristics, especially in its implantation medium (effect of pH, of moisture), structure, texture, mesh size, pore type and density or perforation of the polymer fabric or nonwoven. Another important parameter is the ability of the surfaces of the base polymers forming the matrix or matrices to fix the polymer P (Pᵤ or Pᵣ) in a stable manner. Plasma or corona treatment, or any other physical or chemical surface treatment allowing fixing of the polymer (P) to be increased, can be applied in order to improve its fixing. Preferably, the softening temperature of the matrix (M) must be higher than 70° C. and preferably higher than 90° C. By way of non-limiting examples, woven fabrics and nonwovens based on polypropylenes, polyethylenes, polyamides, polyesters, polyurethanes are particularly suitable for this function.

Preferably, the cross-section dimension of the perforations formed in the base matrix is from 0.2 mm² to 10 mm², especially from 4 mm² to 8 mm².

The thickness of the second material forming the coating of the base matrix is preferably from 500 to 1000 microns, especially from 650 to 750 microns, so that the size or sizes of the perforations in the final product, constituted by the perforated base matrix and the coating, differ(s) very little from that(those) of the base matrix.

Preferably, the polymer (Pᵤ) is chosen from the amorphous or semi-crystalline thermoplastic polymers having a softening temperature (glass transition or melting temperature) higher than 45° C. and, preferably, higher than 50° C. Other parameters for selection are its chemical nature, its biocompatibility, its biodegradability or non-biodegradability in the body, its mechanical and thermomechanical characteristics, especially in the body (effect of pH, of water), its ability to adhere to the surface of M, without modification or after-treatment, and to fix the polymer (Pu) in a stable manner. By way of non-limiting examples there may be mentioned as the second polymer material polymethacrylates, methacrylate-acrylate copolymers, low- and medium-density polyethylenes, ethylene-olefin copolymers, polycaprolactone and its copolymers with lactones, polyethers.

Preferably, the reactive prepolymers (Pᵣ) are chosen from the systems polyol/diisocyanate, polyamine/diisocyanate, the epoxies, and any other polymer carrying reactive functions permitting crosslinking and the formation of a three-dimensional lattice.

Composite products of different shapes can be obtained as a function of the mould used. It is thus possible to produce porous spheres which can be used as ocular prostheses in the case of enucleation or evisceration of the eye.

The present invention relates also to an intraocular ball comprising a composite product of the invention.

It is also possible to produce porous discs which can be used in spinal surgery as an intervertebral disc replacement. Such discs can have various shapes permitting their introduction by simple incision and their easy positioning, but can also have adjusted compressibility, especially by introduction into the core of the disc of a formed element made of metal or of ceramics or of polymer having specific mechanical properties (rigid, flexible, hollow, solid formed element).

It is also possible to use the technique of the present invention to produce prostheses which can be used in facial reconstruction in reparative or aesthetic surgery. Accordingly, it is possible to produce a mould permitting the production of formed elements which can serve as a prosthesis for the chin, the nasal wall or the cheeks. The only limitation in the design of such prostheses being that it is impossible to produce the appropriate shape for the replacement prosthesis by moulding or by hot pressing. The properties of the prostheses can, moreover, readily be altered in terms of rigidity and mechanical strength by the choice of matrix and of coating polymer.

The porosity of these prostheses confers on them great lightness and enables them to be fixed by suturing before the growth of tissue in the open pores ensures perfect cohesion of the prosthesis with its environment.

The present invention relates also to the use of the process according to the invention in the production of an intervertebral disc or an intraocular ball.

The process for the production of the composite products and the characteristics of the composite products are described in detail below by means of a series of examples of different types of application, which are given only by way of illustration.

### EXAMPLES

### 1. Production of Porous Balls Based on a Polypropylene (PP) Matrix Coated with Polymethyl Methacrylate (PMMA)

A 10% by mass solution of PMMA (reference Degacryl 6606F, Degussa) in methyl ethyl ketone (MEK) is prepared in a 2-litre flask, with stirring, and then placed in a large-aperture vessel. A strip of a polypropylene mesh measuring 150 mm×340 mm (reference PPKM802 from Textile Development Associates, TDA) held at both ends is then immersed in the vessel containing the PMMA solution and then dried under a flow of air in order to remove the excess PMMA, which may clog some pores of the mesh. A second immersion of the mesh in the opposite direction to the first is then carried out, followed by drying in a current of air, and the mesh is then dried at 50° C. This procedure can be repeated in order to increase the amount of PMMA fixed to the mesh. Finally, the mesh is dried in an oven in vacuo at 80° C. for 4½ hours. After three immersions, the grammage of the coating is determined by weighing.

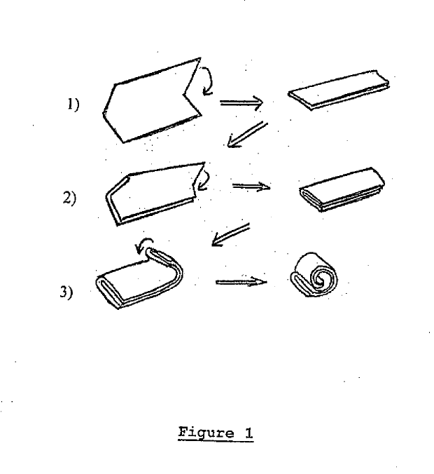

A preformed element measuring 120 mm×90 mm is cut from the coated PP mesh and then folded in accordance with the specific protocol shown in FIG. 1.

Two folds are made in the horizontal direction, then four, still in the horizontal direction. The strip obtained is then rolled up in order to obtain a dense roll of material. This is inserted into the bottom part of a cylindrical mould. The volume of the mould cavity allows the size of the ball to be determined.

The top part of the mould or piston is then inserted and a 360° rotation is applied, then the piston is withdrawn. This action is repeated three times in order to dispose the polymer correctly. Finally, the piston is inserted into the mould conduit and the mould is placed between the two jaws of a heating press, which is brought to 130° C., that is to say to a temperature higher than the softening temperature of the PMMA and lower than that of the polypropylene. The mould is kept hot for 10 minutes and then the mould is allowed to cool to 50° C., after which the device in the form of a ball can be demoulded and recovered.

After demoulding, the balls are immersed in absolute ethanol for one hour and then dried in vacuo for twelve hours in order to remove all traces of volatile compounds.

The porosity of the ball is greater than 50%.

Porosity measurements by mercury porosimetry confirm that the large majority of the pores is accessible from the outside.

These balls are used as an ocular prosthesis. The size and porosity of the balls can be chosen in a broad range allowing especially the needs in terms of ocular prosthesis to be covered.

### 2. Production of Compressible Porous Discs Based on a Polypropylene (PP) Matrix Coated with Polymethyl Methacrylate (PMMA) and Having a Formed Element at the Core

A 10% by mass solution of PMMA (reference Degacryl 6606F, Degussa) in methyl ethyl ketone (MEK) is prepared in a 2-litre flask, with stirring, and then placed in a large-aperture vessel. A preformed element is cut from a polypropylene mesh measuring 120 mm×90 mm (reference PPKM802 from Textile Development Associates, TDA) and a spherical preformed element of PEEK of diameter 5 mm and height 5 mm approximately is placed at its centre and fixed to the mesh.

The assembly constituted by the preformed element to which the disc is attached is then immersed in the vessel containing the PMMA solution and then dried under a flow of air in order to remove the excess PMMA, which may clog some pores of the mesh. A second immersion of the said assembly in the opposite direction to the first is then carried out, followed by drying in a current of air, and the mesh is then dried at 50° C. This procedure can be repeated in order to increase the amount of PMMA fixed to the polypropylene preformed element. Finally, the assembly is dried in an oven in vacuo at 80° C. for 4½ hours.

The coated PP mesh containing the disc is folded in accordance with a specific protocol, so as to hold the central formed element at the core of the polypropylene matrix. The strip obtained is then rolled up in order to obtain a dense roll of material. This is inserted into the bottom part of a cylindrical mould.

The top part of the mould or piston is then inserted into the mould conduit and the mould is placed between the two jaws of a heating press, which is brought to 130° C., that is to say to a temperature higher than or equal to the softening temperature of the PMMA (Tsoft˜100° C.) and lower than that of the polypropylene (Tsoft˜160° C.). The mould is kept hot for ten minutes and then the mould is allowed to cool to 50° C., after which the device in the form of a porous disc can be demoulded and recovered.

After demoulding, the devices are immersed in absolute ethanol for one hour and then dried in vacuo for twelve hours in order to remove all traces of volatile compounds.

### 3. Production of Compressible Porous Discs Based on a Polypropylene Matrix (PP) Coated with Polyurethane (PU)

A multi-hydroxy functional co-oligomer based on MMA and 2-hydroxyethyl methacrylate (HEMA) (average molar mass 1500 g/mol, f_{OH}/=3) synthesised in our laboratories by the radical route is dissolved in methyl ethyl ketone and placed in a 2-litre flask, with stirring, and mixed with hexamethylene diisocyanate in order to have stoichiometry between hydroxyl and isocyanate functions. A strip of a polypropylene mesh measuring 120 mm×90 mm, held at both ends, is then immersed in the vessel containing the mixture. A second immersion of the mesh in the opposite direction to the first is then carried out, followed by evaporation of the MEK at 30° C. in a current of dry air and then under a partial vacuum.

A preformed element is cut from the coated PP mesh and is then folded in accordance with a specific protocol and is inserted into the bottom part of a disc-shaped mould.

The top part of the mould or piston is then inserted into the mould conduit and the mould is placed between the two jaws of a heating press, which is brought to 130° C., that is to say to a temperature higher than the softening temperature of the PU and lower than that of the polypropylene. The mould is kept hot for six hours and then the mould is allowed to cool to 50° C., after which the disc-shaped device can be demoulded and recovered.

After demoulding, the discs are immersed in absolute ethanol for twelve hours in order to remove all traces of unreacted isocyanate functions and then dried in vacuo for twelve hours in order to remove all traces of volatile compounds.

### 4. Production of Compressible Porous Discs Based on a Polypropylene (PP) Matrix Coated with a Reactive Mixture of Monomers

A mixture of monomers, methyl methacrylate (80% by mass), butyl acrylate (15% by mass) and dimethyl methacrylate (3% by mass), and of a radical catalyst, benzoyl peroxide (2% by mass), is placed in a 2-litre flask, with stirring. A preformed element cut from a polypropylene mesh measuring 120 mm×90 mm is then immersed in the vessel containing the mixture. A second immersion of the mesh in the opposite direction to the first is then carried out.

After being drained in order to remove the excess monomers, the coated PP preformed element is inserted into the bottom part of a disc-shaped mould. The top part of the mould is then inserted and the mould is placed between the two jaws of a heating press, which is brought to 100° C., that is to say to a temperature higher than the softening temperature of the mixture of monomers and lower than that of the polypropylene. The mould is kept hot for ten hours and then the mould is allowed to cool to 50° C., after which the disc-shaped device can be demoulded and recovered.

After demoulding, the discs are immersed in absolute ethanol for twelve hours in order to remove all traces of unreacted monomers and then dried in vacuo for twelve hours in order to remove all traces of volatile compounds.

### 5. Production of Compressible Porous Discs Based on a Polypropylene (PP) Matrix Coated with a Reactive Mixture of Monomers Containing a Contrast Agent

A contrast agent, zirconium oxide (25% by mass), is added to a mixture of monomers, identical to that of Example 4 (73% by mass), and of a radical catalyst, benzoyl peroxide (2% by mass), placed in a 2-litre flask, with stirring. A preformed element cut from a polypropylene mesh measuring 120 mm×90 mm is then immersed in the vessel containing the mixture. A second immersion of the mesh in the opposite direction to the first is then carried out.

The coated PP mesh is inserted into the bottom part of a mould. The top part of the mould is then inserted and the mould is placed between the two jaws of a heating press, which is brought to 100° C., that is to say to a temperature higher than the softening temperature of the mixture and lower than that of the polypropylene. The mould is kept hot for ten hours and then the mould is allowed to cool to 50° C., after which the disc-shaped device can be demoulded and recovered.

After demoulding, the discs are immersed in absolute ethanol for twelve hours in order to remove all traces of unreacted monomers and then dried in vacuo for twelve hours in order to remove all traces of volatile compounds.

In Examples 1-5 above, the cross-section dimension of the perforations formed in the mesh is from 0.2 mm² to 10 mm², especially from 4 mm² to 8 mm². The thickness of PMMA is from 700 microns to more or less 50 microns.

In the present description, mention is made of only a first polymer material and a second polymer material. However, each of them can be constituted by a mixture of polymers having characteristics provided for the first polymer and the second polymer, respectively. Accordingly, if the first polymer is constituted by a mixture of N first polymer materials and the second polymer is constituted by a mixture of P second polymer materials, the conditions relating to the softening temperatures become that the highest of the softening or reaction/crosslinking temperatures of the P second polymer materials is lower than the lowest of the softening temperatures of the N first polymer materials and, moreover, the lowest softening temperature of the second polymer materials of type Pᵤ is higher than 45° C. and preferably higher than 50° C.

As defined in the present application, the softening temperature depends upon the grade and the commercial references. A softening range is therefore defined instead, as follows:

There can be used as the first material for the base element, apart from polypropylene (Tsoft from 145° to 175° C.), also high-density polyethylene (Tsoft from 90° to 120° C.), low-density polyethylene (Tsoft from 80° to 115° C.); polyesters such as polyethylene terephthalate (Tsoft from 175° to 210° C.) or polybutylene terephthalate (Tsoft from 180° to 225° C.); or polyamides such as PA 6.6 (Tsoft from 210° to 255° C.), PA 6 (Tsoft from 180 to 220° C.) or PA 11 (Tsoft from 155 to 185° C.).

There can be used as the second material (coating), apart from PMMA, a poly(Σ-caprolactone) (Tsoft from 45 to 50° C.); an ethylene-vinyl acetate copolymer (EVA) (Tsoft from 50 to 70° C., the softening temperature depending upon the ethylene/vinyl acetate ratio, for example for EVATANE 28-800 Tsoft is 63°).

What is claimed is:

1. A composite product, especially an implant, comprising at least one base element or matrix of a first polymer material coated with a coating of a second polymer material, characterised in that the at least one base element or matrix coated with the coating exhibits openings passing through it from one side to the other; and the softening temperature of the second polymer material being lower than the softening temperature of the first polymer material.

2. A composite product, especially an implant, comprising at least one base element or matrix of a first polymer material coated with a coating of a second material based on reactive prepolymers, characterised in that the at least one base element or matrix coated with the coating exhibits openings passing through it from one side to the other; and the reaction/crosslinking temperature of the second material based on reactive prepolymers being lower than the softening temperature of the first polymer material.

3. The composite product according to claim 1, characterised in that the matrix is constituted in the form of a mesh.

4. The composite product according to claim 1, characterised in that the matrix is constituted by a perforated woven sheet.

5. The composite product according to claim 1, characterised in that the matrix is constituted by a perforated nonwoven sheet.

6. The composite product according to claim 1, characterised in that the composite product comprises a plurality of matrices coated with the second polymer material stacked one on top of the other.

7. The composite product according to claim 1, characterised in that the composite product is constituted by a matrix coated with the second polymer material which is folded up, preferably several times.

8. The composite product according to claim 1, characterised in that the plurality of stacked matrices or the folds of the folded matrix are fixed together, especially by hot moulding or thermoforming.

9. The composite product according to claim 8 which is in the form of an intraocular ball.

10. The composite product according to claim 8 which is in the form of an intervertebral disc.

11. A process for the production of a porous composite product, especially according to any one of the preceding claims, especially a porous implant, which comprises steps in which there is taken at least one base element or matrix (M), for example in the form of a mesh, of a perforated woven sheet or a perforated nonwoven sheet of a first polymer material, the base element is coated with a second polymer material (P, Pᵤ; P, Pᵣ); then the coated base element is subjected to hot pressing or thermoforming in order to obtain the composite product, shaping of the composite product by hot moulding or thermoforming being carried out at a temperature higher than the softening temperature of the second polymer material or, in the case of reactive prepolymers, at a temperature higher than their reaction/crosslinking temperature, and at a temperature lower than the softening temperature of the first material, in order thus to conserve its physical, chemical and mechanical integrity.

12. The process according to claim 11, characterised in that a plurality of base matrices of a first polymer material coated with the second material are stacked, and hot pressing is carried out in a mould or hot press, the shape of which corresponds to that of the product that is to be obtained.

13. The process according to claim 11, characterised in that, before the thermoforming step, the coated base element is folded up in a plurality of folds, and then the thermoforming step is carried out in a mould or hot press, the shape of which corresponds to the composite product that is to be obtained.

14. The process according to claim 11 wherein the product is an intervertebral disc.

15. The process according to claim 11 wherein the product is an intraocular ball.

16. The composite product according to claim 2, characterised in that the matrix is constituted in the form of a mesh.

17. The composite product according to claim 2, characterised in that the matrix is constituted by a perforated woven sheet.

18. The composite product according to claim 2, characterised in that the matrix is constituted by a perforated nonwoven sheet.
